(19) United States
(12) Patent Application Publication
(10) Pub. No.: US 2017/0007172 A1
(43) Pub. Date: Jan. 12, 2017

(54) CORRELATION OF SALIVA FLOW-RATE AND ORAL DRYNESS IN HYPERURICEMIA

(71) Applicant: Umm-Al-Qura University, Makkah (SA)

(72) Inventors: Osama Abdulrahman Shaikh-Omar, MAKKAH (SA); ZAHIR Hussain M Akbar, JEDDAH (SA)

(21) Appl. No.: 14/596,194

(22) Filed: Jan. 13, 2015

(51) Int. Cl. A61B 5/00; A61B 10/00; C12Q 1/62

(52) U.S. Cl. CPC A61B 5/4277; C12Q 1/62; A61B 5/4875; A61B 10/0051

(57) ABSTRACT

Hyperuricemia has been studied extensively. However studies on salivary flow rate (SFR; ml/min) in unstimulated saliva (USS) and stimulated saliva (SS) conditions and oral dryness (OD) in subjects with hyperuricemia (HUS) have never been conducted. Current studies showed significantly decreased values in presence of OD (ODP) compared to absence of OD (ODA) in USS both for NUS (normouricemic subjects; p=0.0001) and HUS (p=0.0170). Furthermore, OD comparisons gave significantly decreased values in ODP compared to ODA in SS both for NUS (p=0.0049) and HUS (p=0.0444). In view of the significance in hyperuricemic subjects the “flow rate of unstimulated saliva (USS) and stimulated saliva (SS) decreases significantly in hyperuricemic subjects with oral dryness present (ODP)”. Conclusively, it is suggested that the salivary digestion affected in hyperuricemia can be diagnosed by investigating the salivary composition and flow rate.

### FIELD OF TECHNOLOGY

This disclosure generally relates to an identification of specific markers for the diagnosis of metabolic and digestive disorders. More specifically, this disclosure relates to identification of specific markers for the diagnosis of hyperuricemia.

### BACKGROUND

Presence of excessive uric acid in blood leads to hyperuricemia (HU), which further affects have been found related to gastrointestinal and metabolic disorders (Ktsiki et al, 2013), and it may occur in gastric cancer (Ficarra, 1946), ischemic colitis (IC) in young adult population (Kimura et al, 2012), urinary stones in subjects with a permanent ileostomy (Kennedy et al, 1982), entero-arthrotic syndrome (Angeras, 1961), obstructive uropathy associated with rotavirus gastroenteritis (Ashida et al, 2012), laxative abuse syndrome (Oster et al, 1980), and morbidly obese patients after jejunoileal intestinal bypass surgery (Thomas and Madura, 1997).

Established centuries ago, presence of high levels of uric acid is still considered as the final test to detect HU which is further related to gout. However, the result can sometimes never be so promising which can lead to false or incomplete diagnosis. There is a need for better diagnosis parameters for HU.

### SUMMARY

The present disclosure relates to an identification of a biomarker for a diagnosis of HU. Further, the disclosure also relates to a correlation of saliva flow-rate and oral dryness for the diagnosis of HU. Also, the present disclosure relates to a correlation of an unstimulated and a stimulated saliva flow rate with the oral dryness for the diagnosis of HU. In most embodiments, the present disclosure relates to a correlation and an estimation of the unstimulated saliva flow-rate, stimulated saliva flow-rate, oral dryness and saliva composition as biomarker for the diagnosis of HU.

In one embodiment, the present disclosure relates to estimating a uric acid level in a subject diagnosed with a metabolic disorder. In another embodiment, the present disclosure relates to estimating a serum uric acid level in a subject diagnosed with or prone to having HU. The subject showing serum uric acid level higher than the normal range will be categorized as a HU subject whereas the subject showing serum uric acid level lower than or equal to the normal range will be categorized as a normouricemic subject (NU subject).

In one embodiment, subject categorized as HU subject or NU subject will further be evaluated for an oral dryness. In another embodiment, oral dryness may be evaluated by evaluating lip dryness and/or mouth dryness. In some embodiments, oral dryness may also be evaluated by evaluating cracks on and around the lips. Thus, the subjects diagnosed with HU or NU was further categorized depending on the presence or absence of oral dryness.

In one embodiment, subject was also evaluated according to dietary habit. In another embodiment, subject was evaluated according to the purine rich dietary habit. The subject consuming more purine rich food is more prone to develop metabolic disease such as HU.

In one embodiment, salivary uric acid level will also be estimated in the said subject. In another embodiment, salivary ammonia level along with uric acid level may also be estimated in the said subject. The estimation of salivary uric acid level will also help in diagnosis of HU.

In one embodiment, the present disclosure related to evaluating saliva formation in said subject. In another embodiment, the present disclosure relates to evaluating saliva composition collected from said subject. Saliva may be collected before and after meal or at certain interval of time throughout the day. The collection time of saliva may depend on the subject and the study plan. The saliva composition analysis as disclosed may comprise of estimating water content, an electrolyte such as sodium, potassium, calcium, chlorine, magnesium, bicarbonate, potassium and a protein such as an enzymes along with a immunoglobulin, an antimicrobial factor, a mucosal glycoprotein, an albumin, a polypeptide and an oligopeptide. In some embodiments, antioxidant level will also be evaluated in serum and in saliva.

In one embodiment, saliva from the said subject may be collected in an un-stimulated condition whereas in another embodiment, saliva from said subject may be collected in a stimulated condition to carry further analysis.

In one embodiment, saliva in a stimulated condition may be collected by chewing a paraffin wax whereas in another embodiment, saliva in stimulated condition may be collected by other known methods.

Further, in one embodiment a flow-rate of saliva is also calculated in the said subject. In another embodiment, the flow-rate of saliva is calculated in both stimulated and un-stimulated condition.

Thus, in one embodiment, the present disclosure relates to method 1. A method, comprising: estimating a salivary uric acid level; evaluating an absence or a presence of oral dryness; collecting saliva in a stimulated and an un-stimulated condition; estimating a flow-rate of saliva in the stimulated and un-stimulated condition; and diagnosing a patient with or without hyperuricemia. In another embodiment, the method as disclosed may comprise, admitting subjects prone to HU; estimating serum uric acid level; segregating HU subjects and NU subjects; evaluating absence or presence of oral dryness; collecting saliva in stimulated and un-stimulated condition; estimating flow-rate of saliva; and evaluating subject with HU and NU based on oral dryness and flow-rate of saliva in stimulated and un-stimulated condition. In another embodiment, the method further includes estimating saliva composition and saliva uric acid level in the said subject. In some embodiments, the disclosure further relates to treating subjects diagnosed with hyperuricemia.

Thus, the present disclosure relates to identification of oral dryness, flow-rate of saliva in stimulated and unstimulated conditions as biomarkers for diagnosis of HU whereas in another embodiment, the disclosure relates to estimating saliva composition and saliva uric acid level as a biomarker for a diagnosis of HU.

The Other features will be apparent from the accompanying figures and from the detailed description that follows.

### BRIEF DESCRIPTION OF THE DRAWINGS

Example embodiments are illustrated by way of example and no limitation in the tables and in the accompanying figures, like references indicate similar elements and in which:

FIG. 1 shows a study plan for the present disclosure.

FIG. 2 shows oral dryness and the flow-rate of unstimulated saliva in a HU subject.

FIG. 3 shows oral dryness and the flow-rate of stimulated saliva in a HU subject.

### DETAILED DESCRIPTION

The flow rate of saliva is helpful in understanding the clinical disorders (Humphrey and Williamson, 2001; Dodds et al, 2005), as it plays a prominent role in the lubrication of the alimentary tract bolus, buffering and repairing the oral mucosa, protection against the microorganisms and other oral functions. Increase or decrease in mastication might affect saliva output and may influence in certain medical conditions (Dodds et al, 2005). It was noted that the composition of saliva may play an important role in the perception of food ingredients and liking (Neyrud et al, 2011).Saliva baths the taste receptors and has aroma and taste compounds that are released when food is eaten (Neyrud et al, 2011).

Hyperuricemia (HU) have been found related to gastrointestinal and metabolic disorders (Katsiki et al, 2013), and it may occur in gastric cancer (Ficarra, 1946), ischemic colitis (IC) in young adult population (Kimura et al, 2012), urinary stones in subjects with a permanent ileostomy (Kennedy et al, 1982), entero-arthrotic syndrome (Angeras, 1961), obstructive uropathy associated with rotavirus gastroenteritis (Ashida et al, 2012), laxative abuse syndrome (Oster et al, 1980), and morbidly obese patients after jejunoileal intestinal bypass surgery (Thomas and Madura, 1997).Systemic illnesses are diagnosed by sialometry and sialochemistry for monitoring general health. There are several systemic diseases e.g. coeliac disease (CD) that impair the salivary flow and composition that in turn may cause pathological conditions (Lenander-Lumikari et al, 2000). Saliva is a useful tool in the diagnosis of some physiological and pathological alterations in body functions and in understanding important and interesting aspects of trace metal metabolism (Olmez et al, 1988). There are several factors that can influence salivary secretions and composition. Hence, this requires the collection of standardized saliva that may reflect the real functioning of salivary glands and play role in monitoring the health (Almeida Pdel et al, 2008).

In view of its known involvement in gastrointestinal physiology, the present study was conducted to investigate the influence of salivary flow rate (SFR) and involvement of oral dryness (OD) in NU subject(s) and HU subject(s).

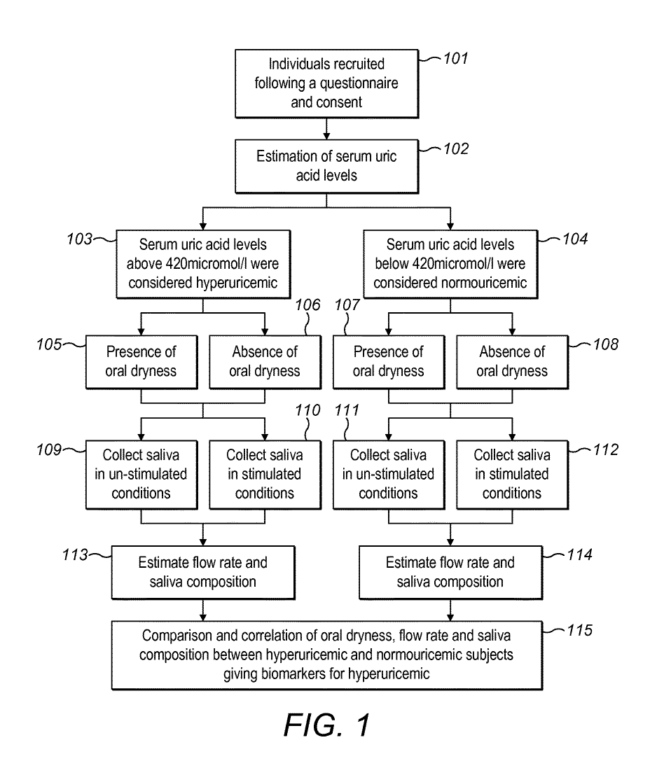

FIG. 1 shows the study plan whereby an individual subject or a plurality of subjects will be recruited in the study plan after getting a questionnaire filled and obtaining their consent 101; serum uric levels will be evaluated from every individual 102; individual subject showing serum uric acid level above the normal range will be categorized as HU subject 103; whereas individual subject showing serum uric acid level below or equal to the normal level will be categorized as NU subject 104; HU and NU subject will further be evaluated for oral dryness as to HU subject showing presence of oral dryness 105 and HU subject showing absence of oral dryness 106; similarly for NU subject showing presence of oral dryness 107 or showing absence of oral dryness 108. Following this, saliva will be calculated from HU subject and NU subject in both stimulated (110, 112) condition and stimulated (109, 111) condition respectively. Saliva flow-rate and saliva composition will then be evaluated in the HU subject 113 and the NU subject 114. The results from oral dryness, flow-rate of saliva in unstimulated and stimulated condition and the saliva composition will then be analyzed and correlated for the biomarker analysis 115.

Thus, the present study comprised 84-100 male subjects without HU (or NU subject) and 72-100 males with HU (HU subject) in the area of Makkah, Kingdom of Saudi Arabia (KSA). The age of the subjects ranged 16-63 years or may depend on the study plan. The selection criteria and other details for those with and without HU as investigated. The HU subject(s) was found to have significantly higher serum uric acid levels compared to NU subject(s). The HU subjects and NU subjects were further categorized into whether they show the presence of oral dryness (ODP) or absence of oral dryness (ODA). The oral dryness (OD) in the current study comprised mainly the lip dryness and was considered as mouth dryness (Shaikh-Omar, 2012). The mean age in both groups showing ODP (NU subject: n: 32; 38.10%; HU subject: 37; 51.39%) and ODA (NU subject: n: 52; 61.90%; HU subject: 35; 48.61%) was quite similar.

A questionnaire was used to collect data on age, sex, educational level, family size, income etc. A food frequency questionnaire was used to illustrate the consumption rate of purine foods (Yeomans, 1991). Uric acid was determined according to the enzymatic colorimetric test. Estimation of uric acid was employed to categorize the subjects into those with and others without HU. Whether a subject be included in the HU group or not is also based on their consumption of purine-rich diets and confirmed by estimating the serum uric acid levels. The subjects showing serum uric acid level above 420 micromol/l for males were considered to be included in HU subject group (Al-Arfaj, 2001).

The saliva from both groups in unstimulated condition was collected in milliliters for five minutes duration and evaluated as ml/min. The collection of saliva in stimulated condition was also carried out. Chewing the paraffin-wax was used for collecting the samples of stimulated saliva. The stimulated saliva and unstimulated saliva were collected during 9-10 AM or other time as decided during the study plan. Salivary flow rate (saliva in ml/min; SFR) was evaluated in both conditions i.e stimulated and unstimulated condition. Saliva was then centrifuged and measurements were carried out by routine kit methods. The values were denoted as mean ±SEM or SD. The analyzed data is given in Table 1 and 2. Statistical analysis of the data was done simply by employing students' unpaired t-test using SPSS program, and values of ‘p’ were found.

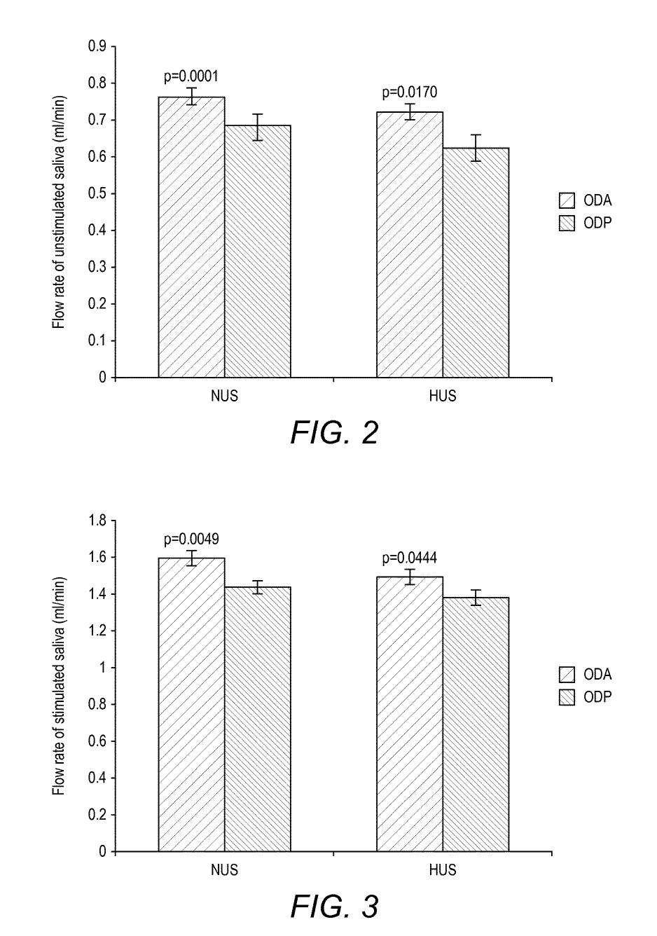

In the disclosed studies the NU subject(s) and HU subject(s) respectively showed ODP as 38.10% (n=32) and 51.39% (n=37). Whereas the ODA in NU subject(s) and HU subject(s) respectively was 61.9% (n=52) and 48.61% (n=35). The mean±SEM values for SFR for unstimulated saliva (Table 1) and stimulated saliva are given in Table 1 and 2.The ODP vs ODA in both NU subject(s) and HU subject(s) were found significantly different (FIG. 2). The mean±SEM values for SFR for unstimulated saliva (Table 1) showed non-significant decrease in HU subject(s) compared to NU subject(s) in both ODP and ODA. However, the OD comparisons revealed significantly decreased values in ODP compared to ODA both for NU subject(s) (p=0.0001) and HU subject(s) (p=0.0170) (FIG. 2).

TABLE 1
Flow rate of unstimulated saliva in hyperuricemic male Saudis.

| Physiological | Salivary flow rate (ml/min) | | | Significance |
|---|---|---|---|---|
| Conditions | | NUS (n: 84) | HUS (n: 72) | (p) |
| ODP | Mean | 0.68 | 0.62 | 0.2350 |
| | SEM | 0.0355 (32) | 0.0350 (37) | |
| ODA | Mean | 0.76 | 0.72 | 0.1906 |
| | SEM | 0.0209 (52) | 0.0200 (35) | |

TABLE 2
Flow rate of stimulated saliva in hyperuricemic male Saudis.

| Physiological | Salivary flow rate (ml/min) | | | Significance |
|---|---|---|---|---|
| Conditions | | NUS (n: 84) | HUS (n: 72) | (p) |
| ODP | Mean | 1.44 | 1.38 | 0.3132 |
| | SEM | 0.0421 (32) | 0.0411 (37) | |
| ODA | Mean | 1.60 | 1.49 | 0.0333 |
| | SEM | 0.0348 (52) | 0.0341 (35) | |

The mean±SEM values for SFR for stimulates saliva (Table 2) gave non-significant decrease in HU subject(s) compared to NU subject(s) in both ODP and ODA. However, the OD comparisons indicated significantly decreased values in ODP compared to ODA both for NU subject(s) (p=0.0049) and HU subject(s) (p=0.0444) (FIG. 3).

In some embodiments, saliva composition analysis is also performed which may comprise (but not limited to) estimating water content, an electrolyte such as sodium, potassium, calcium, chlorine, magnesium, bicarbonate, potassium and a protein such as an enzymes along with a immunoglobulin, an antimicrobial factor, a mucosal glycoprotein, an albumin, a polypeptide and a oligopeptide. Levels of antioxidants may also be evaluated in blood serum and in saliva in both HU subject(s) and NU subjects(s).

Thus, the present study depicts for the first time a novel correlation between salivary uric acid level, flow-rate of unstimulated and stimulated saliva and oral dryness as biomarkers for the diagnosis of HU. Furthermore, this will also lead to better treatment procedures as compared to presently used methods.

What is claimed is:

1. A method to identify hyperuricemia in a subject, the method comprising:
estimating a uric acid level of the subject in need of diagnosis for the hyperuricemia, the uric acid level being measured in a saliva or blood;
categorizing the subject as hyperuricemic when the uric acid level is above 420 micromol/l and as normouricemic when the uric acid level is below 420 micromol/l;
evaluating an absence or a presence of oral dryness of the subject;
collecting an unsimulated saliva and a simulated saliva of the subject;
estimating a flow-rate of the unsimulated saliva and a simulated saliva, the flow-rate being estimated based on the collected the unsimulated saliva and the simulated saliva; and
comparing oral dryness, the flow rate of an unsimulated saliva and a simulated saliva, and the hyperurecimia in a subject to diagnose hyperuricemic state in a subject.

2. The method of claim 1, further comprising evaluating salivary composition for diagnosis of the subject with or without hyperuricemia.

3. The method of claim 1, wherein oral dryness being evaluated by evaluating lip dryness and/or mouth dryness.

4. The method of claim 1, wherein the uric acid level is estimated in the blood serum from a subject.

5. The method of claim 1, wherein the uric acid level is estimated in the saliva of a subject.

6. The method of claim 5, wherein uric acid level in saliva is greater in the subject with hyperuricemia as compared to normal human without hyperuricemia.

7. The method of claim 1, wherein the saliva is collected in both an un-stimulated condition and a stimulated condition.

8. The method of claim 7, wherein the saliva is collected in the stimulated condition by chewing a paraffin wax.

9. (canceled)

10. The method of claim 1, wherein the subject diagnosed with the hyperuricemia will further be subjected to treatment of the hyperuricemia.
